(19) United States
(12) Patent Application Publication
(10) Pub. No.: US 2024/0415843 A1
(43) Pub. Date: Dec. 19, 2024

(54) USES OF TRI-SUBSTITUTED HETEROARYL DERIVATIVES AS SRC HOMOLOGY-2 PHOSPHATASE INHIBITORS

(71) Applicant: Erasca, Inc., San Diego, CA (US)

(72) Inventors: Leslie Harris BRAIL, San Diego, CA (US); Wei LIN, San Diego, CA (US); Dawei XUAN, San Diego, CA (US); Robert F. SHOEMAKER, San Diego, CA (US); Leenus MARTIN, San Diego, CA (US)

(21) Appl. No.: 18/698,492

(22) PCT Filed: Oct. 6, 2022
(86) PCT No.: PCT/US2022/045856

§ 371 Date: Apr. 4, 2024

Related U.S. Application Data
(60) Provisional application No. 63/253,003, filed on Oct. 6, 2021.

(51) Int. Cl. A61K 31/5383; A61K 9/00; A61K 9/20; A61K 9/48; A61P 35/00

(52) U.S. Cl. CPC A61K 31/5383; A61K 9/0019; A61K 9/2013; A61K 9/2018; A61K 9/2054; A61K 9/2059; A61K 9/4858; A61P 35/00

(57) ABSTRACT

The present disclosure provides certain tri-substituted heteroaryl derivatives that are Src Homology-2 phosphatase (SHP2) inhibitors and are therefore useful for the treatment of diseases treatable by inhibition of SHP2. Also provided are pharmaceutical compositions containing such compounds and uses of such compounds.

This application claims the benefit of U.S. Provisional Application Ser. No. 63/253,003 filed Oct. 6, 2021 which is hereby incorporated by reference in its entirety.

### BACKGROUND

Src Homology-2 phosphatase (SHP2) is a non-receptor protein phosphatase ubiquitously expressed in various tissues and cell types (see reviews: Tajan M et al., Eur J Med Genet 2016 58 (10): 509-25; Grossmann K S et al., Adv Cancer Res 2010 106:53-89). SHP2 is composed of two Src homology 2 (N—SH2 and C—SH2) domains in its NH2-terminus, a catalytic PTP (protein-tyrosine phosphatase) domain, and a C-terminal tail with regulatory properties. At the basal state, the intermolecular interactions between the SH2 domains and the PTP domain prevent the access of substrates to the catalytic pocket, keeping SHP2 into a closed, auto-inhibited conformation. In response to stimulation, SHP2 activating proteins bearing phosphor-tyrosine motifs bind to the SH2 domains, leading to exposure of active site and enzymatic activation of SHP2.

### SUMMARY

The present disclosure provides certain tri-substituted heteroaryl derivatives that are Src Homology-2 phosphatase (SHP2) inhibitors and are therefore useful for the treatment of diseases treatable by inhibition of SHP2.

Disclosed herein is a method of treating a disease treatable by inhibition of SHP2 in a patient, comprising administering to the patient a therapeutically effective amount of Compound I:

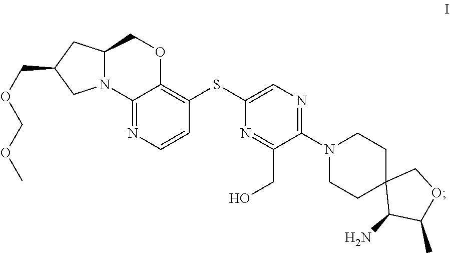 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof.

Also disclosed herein is a method of treating a disease treatable by inhibition of SHP2 in a patient, comprising administering to the patient a therapeutically effective amount of Compound I:

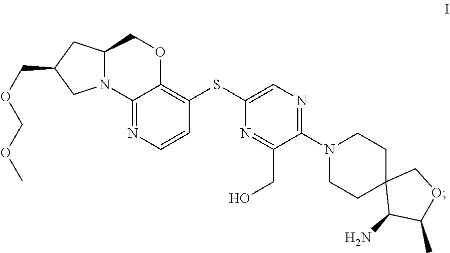 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; wherein the compound or pharmaceutically acceptable salt thereof, is administered in an amount of 20 mg to 120 mg once a day for two weeks, followed by a one week break in at least one three-week cycle.

Also disclosed herein is a method of treating a disease treatable by inhibition of SHP2 in a patient, comprising administering to the patient a therapeutically effective amount of Compound I:

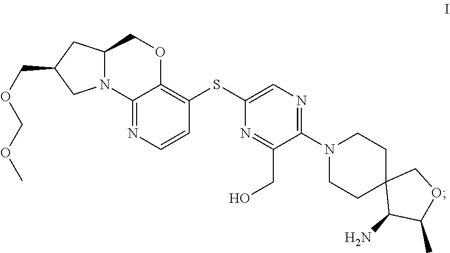 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; wherein the compound or pharmaceutically acceptable salt thereof, is administered in an amount of 20 mg to 120 mg once a day for three weeks, followed by a one week break in at least one four-week cycle.

In some embodiments, the compound or pharmaceutically acceptable salt thereof, is administered once a day in an amount of 20 mg to 60 mg. In some embodiments, the compound or pharmaceutically acceptable salt thereof, is administered once a day in an amount of 20 mg to 40 mg. In some embodiments, the compound or pharmaceutically acceptable salt thereof, is administered once a day in an amount of 20 mg. In some embodiments, the compound or pharmaceutically acceptable salt thereof, is administered once a day in an amount of 25 mg. In some embodiments, the compound or pharmaceutically acceptable salt thereof, is administered once a day in an amount of 30 mg. In some embodiments, the compound or pharmaceutically acceptable salt thereof, is administered once a day in an amount of 35 mg. In some embodiments, the compound or pharmaceutically acceptable salt thereof, is administered once a day in an amount of 40 mg. In some embodiments, the compound or pharmaceutically acceptable salt thereof, is administered once a day in an amount of 45 mg. In some embodiments, the compound or pharmaceutically acceptable salt thereof, is administered once a day in an amount of 50 mg. In some embodiments, the compound or pharmaceutically acceptable salt thereof, is administered once a day in an amount of 55 mg. In some embodiments, the compound or pharmaceutically acceptable salt thereof, is administered once a day in an amount of 60 mg. In some embodiments, the compound or pharmaceutically acceptable salt thereof, is administered once a day in an amount of 65 mg. In some embodiments, the compound or pharmaceutically acceptable salt thereof, is administered once a day in an amount of 70 mg. In some embodiments, the compound or pharmaceutically acceptable salt thereof, is administered once a day in an amount of 75 mg. In some embodiments, the compound or pharmaceutically acceptable salt thereof, is administered once a day in an amount of 80 mg. In some embodiments, the compound or pharmaceutically acceptable salt thereof, is administered once a day in an amount of 85 mg. In some embodiments, the compound or pharmaceutically acceptable salt thereof, is administered once a day in an amount of 90 mg. In some embodiments, the compound or pharmaceutically acceptable salt thereof, is administered once a day in an amount of 95 mg. In some embodiments, the compound or pharmaceutically acceptable salt thereof, is administered once a day in an amount of 100 mg. In some embodiments, the compound or pharmaceutically acceptable salt thereof, is administered once a day in an amount of 105 mg. In some embodiments, the compound or pharmaceutically acceptable salt thereof, is administered once a day in an amount of 110 mg. In some embodiments, the compound or pharmaceutically acceptable salt thereof, is administered once a day in an amount of 115 mg. In some embodiments, the compound or pharmaceutically acceptable salt thereof, is administered once a day in an amount of 120 mg. In some embodiments, the compound or pharmaceutically acceptable salt thereof is administered for at least two three- or four-week cycles. In some embodiments, the compound or pharmaceutically acceptable salt thereof is administered for at least three three- or four-week cycles. In some embodiments, the compound or pharmaceutically acceptable salt thereof is administered for at least four three- or four-week cycles. In some embodiments, the compound or pharmaceutically acceptable salt thereof is administered for at least five three- or four-week cycles. In some embodiments, the compound or pharmaceutically acceptable salt thereof is administered for at least six three- or four-week cycles. In some embodiments, the compound or pharmaceutically acceptable salt thereof, is formulated as a pharmaceutical composition. In some embodiments, the compound or pharmaceutically acceptable salt thereof, is formulated as an oral composition. In some embodiments, the disease is cancer. In some embodiments, the cancer is colorectal cancer, lung cancer, non-small cell lung cancer, stomach cancer, liver cancer, colon cancer, kidney cancer, breast cancer, head and neck cancer, head and neck squamous cell carcinoma, endometrial carcinoma, pancreatic cancer, pancreatic ductal adenocarcinoma, melanoma, liposarcoma, neuroblastoma, juvenile myelomonocytic leukemia, or acute myeloid leukemia. In some embodiments, the disease is Noonan syndrome or Leopard syndrome.

Also disclosed herein is a method of treating a disease treatable by inhibition of SHP2 in a patient, comprising administering to the patient a therapeutically effective amount of Compound I:

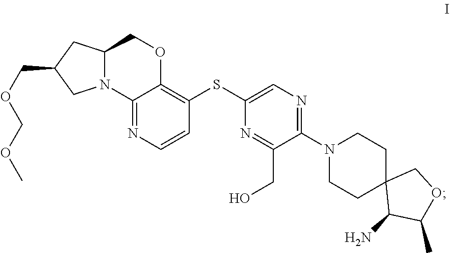 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; wherein the compound or pharmaceutically acceptable salt thereof, is administered in an amount of 40 mg to 120 mg twice a day for two weeks followed by a one week break in at least one three-week cycle.

Also disclosed herein is a method of treating a disease treatable by inhibition of SHP2 in a patient, comprising administering to the patient a therapeutically effective amount of Compound I:

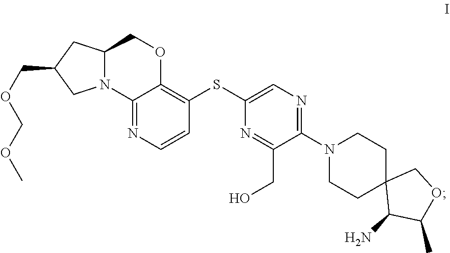 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; wherein the compound or pharmaceutically acceptable salt thereof, is administered in an amount of 40 mg to 120 mg twice a day for three weeks, followed by a one week break in at least one four-week cycle.

In some embodiments, the compound or pharmaceutically acceptable salt thereof, is administered twice a day in an amount of 40 mg to 80 mg. In some embodiments, the compound or pharmaceutically acceptable salt thereof, is administered twice a day in an amount of 40 mg. In some embodiments, the compound or pharmaceutically acceptable salt thereof, is administered twice a day in an amount of 45 mg. In some embodiments, the compound or pharmaceutically acceptable salt thereof, is administered twice a day in an amount of 50 mg. In some embodiments, the compound or pharmaceutically acceptable salt thereof, is administered twice a day in an amount of 55 mg. In some embodiments, the compound or pharmaceutically acceptable salt thereof, is administered twice a day in an amount of 60 mg. In some embodiments, the compound or pharmaceutically acceptable salt thereof, is administered twice a day in an amount of 65 mg. In some embodiments, the compound or pharmaceutically acceptable salt thereof, is administered twice a day in an amount of 70 mg. In some embodiments, the compound or pharmaceutically acceptable salt thereof, is administered twice a day in an amount of 75 mg. In some embodiments, the compound or pharmaceutically acceptable salt thereof, is administered twice a day in an amount of 80 mg. In some embodiments, the compound or pharmaceutically acceptable salt thereof, is administered twice a day in an amount of 85 mg. In some embodiments, the compound or pharmaceutically acceptable salt thereof, is administered twice a day in an amount of 90 mg. In some embodiments, the compound or pharmaceutically acceptable salt thereof, is administered twice a day in an amount of 95 mg. In some embodiments, the compound or pharmaceutically acceptable salt thereof, is administered twice a day in an amount of 100 mg. In some embodiments, the compound or pharmaceutically acceptable salt thereof, is administered twice a day in an amount of 105 mg. In some embodiments, the compound or pharmaceutically acceptable salt thereof, is administered twice a day in an amount of 110 mg. In some embodiments, the compound or pharmaceutically acceptable salt thereof, is administered twice a day in an amount of 115 mg. In some embodiments, the compound or pharmaceutically acceptable salt thereof, is administered twice a day in an amount of 120 mg. In some embodiments, the compound or pharmaceutically acceptable salt thereof is administered for at least two three- or four-week cycles. In some embodiments, the compound or pharmaceutically acceptable salt thereof is administered for at least three three- or four-week cycles. In some embodiments, the compound or pharmaceutically acceptable salt thereof is administered for at least four three- or four-week cycles. In some embodiments, the compound or pharmaceutically acceptable salt thereof is administered for at least five three- or four-week cycles. In some embodiments, the compound or pharmaceutically acceptable salt thereof is administered for at least six three- or four-week cycles. In some embodiments, the compound or pharmaceutically acceptable salt thereof, is formulated as a pharmaceutical composition. In some embodiments, the compound or pharmaceutically acceptable salt thereof, is formulated as an oral composition. In some embodiments, the disease is cancer. In some embodiments, the cancer is colorectal cancer, lung cancer, non-small cell lung cancer, stomach cancer, liver cancer, colon cancer, kidney cancer, breast cancer, head and neck cancer, head and neck squamous cell carcinoma, endometrial carcinoma, pancreatic cancer, pancreatic ductal adenocarcinoma, melanoma, liposarcoma, neuroblastoma, juvenile myelomonocytic leukemia, or acute myeloid leukemia. In some embodiments, the disease is Noonan syndrome or Leopard syndrome.

### DETAILED DESCRIPTION

The present embodiments provide methods of treating a disease treatable by inhibition of SHP2 in a patient, comprising administering to the patient a therapeutically effective amount of Compound I:

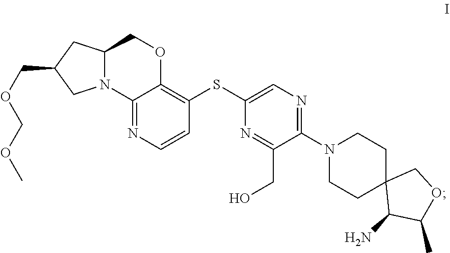 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof.

In some embodiments, the compound or pharmaceutically acceptable salt thereof is formulated as a pharmaceutical composition. In some embodiments, the compound or pharmaceutically acceptable salt thereof is formulated as an oral composition.

In some embodiments, the compound or pharmaceutically acceptable salt thereof is administered once or twice a day. In some embodiments, the compound or pharmaceutically acceptable salt thereof is administered once a day. In some embodiments, the compound or pharmaceutically acceptable salt thereof is administered twice a day.

In some embodiments, the compound or pharmaceutically acceptable salt thereof is administered over a continuous 28-day cycle.

In some embodiments, the compound or pharmaceutically acceptable salt thereof is administered once a day in the amount of 10 mg to 140 mg. In some embodiments, the compound or pharmaceutically acceptable salt thereof is administered once a day in the amount of 20 mg to 80 mg. In some embodiments, the compound or pharmaceutically acceptable salt thereof is administered once a day in the amount of 20 mg to 60 mg. In some embodiments, the compound or pharmaceutically acceptable salt thereof is administered once a day in the amount of 20 mg to 40 mg. In some embodiments, the compound or pharmaceutically acceptable salt thereof is administered once a day in the amount of 20 mg to 120 mg.

In some embodiments, the compound or pharmaceutically acceptable salt thereof is administered twice day in the amount of 10 mg to 100 mg. In some embodiments, the compound or pharmaceutically acceptable salt thereof is administered twice day in the amount of 40 mg to 120 mg. In some embodiments, the compound or pharmaceutically acceptable salt thereof is administered twice day in the amount of 40 mg to 80 mg. In some embodiments, the compound or pharmaceutically acceptable salt thereof is administered twice day in the amount of 20 mg to 60 mg. In some embodiments, the compound or pharmaceutically acceptable salt thereof is administered twice day in the amount of 20 mg to 40 mg.

In some embodiments, the compound or pharmaceutically acceptable salt thereof is administered once a day for two weeks, followed by a one week break over a period of 3 weeks. In some embodiments, the compound or pharmaceutically acceptable salt thereof is administered once a day for three weeks, followed by a one week break over a period of 4 weeks.

In some embodiments, the compound or pharmaceutically acceptable salt thereof is administered twice a day for two weeks, followed by a one week break over a period of 3 weeks. In some embodiments, the compound or pharmaceutically acceptable salt thereof is administered twice a day for three weeks, followed by a one week break over a period of 4 weeks.

In some embodiments, the compound or pharmaceutically acceptable salt thereof is administered over a period of 6 weeks. In some embodiments, the compound or pharmaceutically acceptable salt thereof is administered over a period of 8 weeks.

In some embodiments, the compound or pharmaceutically acceptable salt thereof is administered 3 times a week. In some embodiments, the compound or pharmaceutically acceptable salt thereof is administered on day 1, day 3, and day 5 of the week.

In some embodiments, the compound or pharmaceutically acceptable salt thereof is administered 4 times a week. In further embodiments, the compound or pharmaceutically acceptable salt thereof is administered 4 times a week for two weeks, followed by a one week break over a period of 3 weeks. In further embodiments, the compound or pharmaceutically acceptable salt thereof is administered 4 times a week for three weeks, followed by a one week break over a period of 4 weeks.

In some embodiments, the compound or pharmaceutically acceptable salt thereof is administered twice a day, two days per week.

In some embodiments, the compound or pharmaceutically acceptable salt thereof is administered over a period of 8 weeks.

In some embodiments, the compound or pharmaceutically acceptable salt thereof is administered on day 1 and day 2 of each week.

In some embodiments, the disease treatable by inhibition of SHP2 is cancer. In further embodiments, the cancer is colorectal cancer, lung cancer, non-small cell lung cancer, stomach cancer, liver cancer, colon cancer, kidney cancer, breast cancer, head and neck cancer, head and neck squamous cell carcinoma, endometrial carcinoma, pancreatic cancer, pancreatic ductal adenocarcinoma, melanoma, liposarcoma, neuroblastoma, juvenile myelomonocytic leukemia, or acute myeloid leukemia. In some embodiments, the disease treatable by inhibition of SHP2 is Noonan syndrome or Leopard syndrome.

In a related aspect, the present disclosure provides a use of pharmaceutical compound comprising a therapeutically effective amount of Compound I:

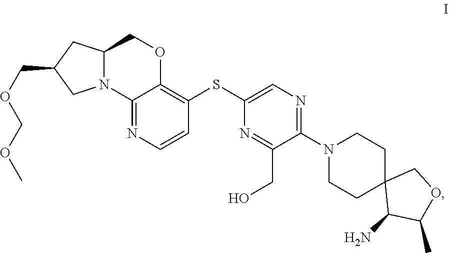 [SMILES: COCOC[C@H]1C[C@H]2COc3c(Sc4cnc(N5CCC6(CC5)CO[C@@H](C)[C@H]6N)c(CO)n4)ccnc3N2C1]

or pharmaceutically acceptable salt thereof;
for the treatment of a disease treatable by inhibition of SHP2 in a patient.

### Definitions

Unless specifically indicated otherwise, all technical and scientific terms used herein have the same meaning as commonly understood by those of ordinary skill in the art to which the embodiments are directed. In addition, any method or material similar or equivalent to a method or material described herein can be used in the practice of the embodiments herein. For purposes of the embodiments disclosed herein, the following terms are defined.

“A,” “an,” or “the” as used herein not only include aspects with one member, but also include aspects with more than one member. For instance, the singular forms “a.” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass±10%, preferably ±5%, the recited value and the range is included.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject. In the context of the combination therapies disclosed herein, administration can be at separate times or simultaneous or substantially simultaneous.

“Therapeutically effective amount” refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins), each of which is incorporated herein by reference in its entirety for all of its teachings, including without limitation all methods, compounds, compositions, data and the like, for use with any of the embodiments and disclosure herein. In sensitized cells, the therapeutically effective dose can often be lower than the conventional therapeutically effective dose for non-sensitized cells.

“Pharmaceutically acceptable excipient” refers to a substance that aids the administration of an active agent to and absorption by a subject. Pharmaceutical excipients useful in the present embodiments include, but are not limited to, binders, fillers, disintegrants, lubricants, surfactants, coatings, sweeteners, flavors, and colors. One of skill in the art will recognize that other pharmaceutical excipients are useful in the present embodiments.

“Treat,” “treating” and “treatment” refer to any indicia of success in the treatment or amelioration of an injury, pathology, or condition, including any objective or subjective parameter such as abatement: remission: diminishing of symptoms or making the injury, pathology or condition more tolerable to the patient: slowing in the rate of degeneration or decline: making the final point of degeneration less debilitating: improving a patient's physical or mental well-being. The treatment or amelioration of symptoms can be based on objective or subjective parameters: including the results of a physical examination, neuropsychiatric exams, and/or a psychiatric evaluation.

“Subject” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, horse, and other non-mammalian animals. In some embodiments, the patient is human.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

“Inhibition,” “inhibits” and “inhibitor” refer to a compound that partially or completely blocks or prohibits or a method of partially or fully blocking or prohibiting, a specific action or function.

“Cancer” refers to all types of cancer, neoplasm or malignant tumors found in mammals (e.g. humans), including, without limitation, leukemias, lymphomas, carcinomas, and sarcomas. Exemplary cancers that may be treated with a compound or method provided herein include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

“EGFR inhibitor” refers to any inhibitor of wild-type EGFR or an EGFR mutant. EGFR mutations include, but are not limited to, any of those disclosed in U.S. Patent Publication No. 2018/0235968, which is incorporated herein by reference in its entirety. EGFR mutations include, without limitation, single nucleotide polymorphisms, exon insertion and deletions, polysomy, and the like. Specific examples of mutations include, without limitation, EGFR gene copy gain, EGFR gene amplification, chromosome 7 polysomy, EGFR L858R, EGFR exon 19 deletions/insertions (e.g., E746_A750del, E746_T751delinsI, E746_T751delinsIP, E746_S752delinsA, E746_S752delinsV. E746_S752delinsV. L747_S752del, L747_T751del, and L747_P753delinsS), EGFR L861Q, EGFR G719C, EGFR G719S, EGFR G719A. EGFR V765A, EGFR T783A, EGFR exon 20 insertions (e.g., N771dup. N771_H773dup, and P772_H773dup), EGFR splice variants (e.g., Viii, Vvi, and Vii), EGFR A289D, EGFR A289T, EGFR A289V, EGFR G598A, EGFR G598V. EGFR T790M, and EGFR C797S. In some embodiments, one or more of the mutations listed in this paragraph and elsewhere herein can be specifically excluded from the embodiments set forth herein, including without limitation, any methods, kits, and compositions of matter, etc. Non-limiting examples of EGFR inhibitors include osimertinib, dacomitinib, lazertinib, nazartinib, neratinib, mobocertinib, afatinib, erlotinib, gefitinib, lapatinib, lifirafenib, amivantamab, cetuximab, panitumumab, necitumumab, mirzotamab clezutoclax, nimotuzumab and vandetanib. Other EGFR inhibitors include those disclosed in U.S. Patent Publication Nos. 2020/0002279, 2019/0202920 and 2019/0167686 and International applications WO2012/061299, WO2019/067543, WO2020/190765, each of which are incorporated herein by reference in their entirety. In some embodiments, one or more of the inhibitors listed in this paragraph and elsewhere herein, and those in the incorporated references, can be specifically excluded from one or more of the embodiments set forth herein, including without limitation, any methods, kits, and compositions of matter, etc.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

### Methods of Treatment

Disclosed herein is a method of treating a disease treatable by inhibition of SHP2, the method comprising administering to a subject or patient a therapeutically effective amount of Compound I or pharmaceutically acceptable salt thereof.

The Src Homolgy-2 phosphatase (SHP2) is a protein tyrosine phosphatase encoded by the PTPNl 1 gene that contributes to multiple cellular functions including proliferation, differentiation, cell cycle maintenance and migration. SHP2 is involved in signaling through the Ras-mitogen-activated protein kinase, the JAK-STAT or the phosphoinositol 3-kinase-AKT pathways. SHP2 mediates activation of Erk1 and Erk2 (Erk1/2, Erk) MAP kinases by receptor tyrosine kinases such as ErbB1, ErbB2 and c-Met.

SHP2 has two N-terminal Src homology 2 domains (N—SH2 and C—SH2), a catalytic domain (PTP), and a C-terminal tail. The two SH2 domains control the subcellular localization and functional regulation of SHP2. The molecule exists in an inactive conformation, inhibiting its own activity via a binding network involving residues from both the N—SH2 and PTP domains. In response to growth factor stimulation, SHP2 binds to specific tyrosine-phosphorylated sites on docking proteins such as Gab1 and Gab2 via its SH2 domains. This induces a conformational change that results in SHP2 activation.

Mutations in PTPN11 have been identified in several human diseases, such as Noonan Syndrome. Leopard Syndrome, juvenile myelomonocytic leukemias, neuroblastoma, melanoma, acute myeloid leukemia, and cancers of the breast, lung, and colon. SHP2 is an important downstream signaling molecule for a variety of receptor tyrosine kinases, including the receptors of platelet-derived growth factor (PDGF-R), fibroblast growth factor (FGF-R) and epidermal growth factor (EGF-R). SHP2 is also an important downstream signaling molecule for the activation of the mitogen activated protein (MAP) kinase pathway which can lead to cell transformation, a prerequisite for the development of cancer. Knock-down of SHP2 significantly inhibited cell growth of lung cancer cell lines with SHP2 mutation or EML4/ALK translocations as well as EGFR amplified breast cancers and esophageal cancers. SHP2 is also activated downstream of oncogenes in gastric carcinoma, anaplastic large-cell lymphoma and glioblastoma.

Noonan Syndrome (NS) and Leopard Syndrome (LS): PTPN11 mutations cause LS (multiple lentigenes, electrocardiogramduction abnormalities, ocular hypertelorism, pulmonic stenosis, abnormal genitalia, retardation of growth, sensorineural deafness) and NS (congenital anomalies including cardiac defects, craniofacial abnormalities, and short stature). Both disorders are part of a family of autosomal dominant syndromes caused by germline mutations in components of the RAS/RAF/MEK/ERK mitogen activating protein kinase pathway, required for normal cell growth and differentiation. Aberrant regulation of this pathway has profound effects, particularly on cardiac development, resulting in various abnormalities, including valvuloseptal defects and/or hypertrophic cardiomyopathy (HCM). Perturbations of the MAPK signaling pathway have been established as central to these disorders and several candidate genes along this pathway have been identified in humans, including mutations in KRAS, NRAS, SOS1, RAFI, BRAF, MEK1, MEK2, SHOC2, and CBL. The gene most commonly mutated in NS and LS is PTPNl 1. Germline mutations in PTPNl 1 (SHP2) are found in-50% of the cases with NS and nearly all patients with LS that shares certain features with NS. For NS, Y62D and Y63C substitutions in the protein are largely invariant and are among the most common mutations. Both these mutations affect the catalytically inactive conformation of SHP2 without perturbing the binding of the phosphatase to its phosphorylated signaling partners.

Juvenile Myelomonocytic Leukemias (JMML):—Somatic mutations in PTPNl 1 (SHP2) occur in about 35% of the patients with JMML, a childhood myeloproliferative disorder (MPD). These gain-of-function mutations are typically point mutations in the N—SH2 domain or in the phosphatase domain, which prevent self-inhibition between the catalytic domain and the N—SH2 domain, resulting in SHP2 activity.

Acute Myeloid Leukemia: PTPNl 1 mutations have been identified in: −10% of pediatric acute leukemias, such as myelodysplastic syndrome (MDS); −7% of B cell acute lymphoblastic leukemia (B-ALL); and −4% of acute myeloid leukemia (AML).

NS and leukemia mutations cause changes in amino acids located at the interface formed by the N—SH2 and PTP domains in the self-inhibited SHP2 conformation, disrupting the inhibitory intramolecular interaction, leading to hyperactivity of the catalytic domain.

SHP2 acts as a positive regulator in receptor tyrosine kinase (RTK) signaling. Cancers containing RTK alterations (EGFR amp, Her2 amp, FGFR amp, Met 31″15, translocated/activated RTK, i.e. ALK, BCR/ABL) include Esophageal, Breast, Lung, Colon, Gastric, Glioma, Head and Neck cancers.

Esophageal cancer (or esophageal cancer) is a malignancy of the esophagus. There are various subtypes, primarily squamous cell cancer (<50%) and adenocarcinoma. There is a high rate of RTK expression in esophageal adenocarcinoma and squamous cell cancer. A SHP2 inhibitor of the invention can, therefore, be employed for innovative treatment strategies.

Breast cancer is a major type of cancer and a leading cause of death in women, where patients develop resistance to current drugs. There are four major subtypes of breast cancers including luminal A, luminal B, Her2 like, and triple negative/Basal-like. Triple negative breast cancer (TNBC) is an aggressive breast cancer lacking specific targeted therapy. Epidermal growth factor receptor I (EGFR) has emerged as a promising target in TNBC. Inhibition of Her2 as well as EGFR via SHP2 may be a promising therapy in breast cancer.

Lung Cancer-NSCLC is currently a major cause of cancer-related mortality, accounting for about 85% of lung cancers (predominantly adenocarcinomas and squamous cell carcinomas). Although cytotoxic chemotherapy remains an important part of treatment, targeted therapies based on genetic alterations such as EGFR and ALK in the tumor are more likely to benefit from a targeted therapy.

Colon Cancer-Approximately 30% to 50% of colorectal tumors are known to have a mutated (abnormal) KRAS, and BRAF mutations occur in 10 to 15% of colorectal cancers. For a subset of patients whose colorectal tumors have been demonstrated to over express EGFR, these patients exhibit a favorable clinical response to anti-EGFR therapy.

Gastric Cancer is one of the most prevalent cancer types. Aberrant expression of tyrosine kinases, as reflected by the aberrant tyrosine phosphorylation in gastric cancer cells, is known in the art. Three receptor-tyrosine kinases, c-met (HGF receptor), FGF receptor 2, and erbB2/neu are frequently amplified in gastric carcinomas. Thus, subversion of different signal pathways may contribute to the progression of different types of gastric cancers.

Neuroblastoma is a pediatric tumor of the developing sympathetic nervous system, accounting for about 8% of childhood cancers. Genomic alterations of the anaplastic lymphoma kinase (ALK) gene have been postulated to contribute to neuroblastoma pathogenesis.

Squamous-cell carcinoma of the head and neck (SCCHN). High levels of EGFR expression are correlated with poor prognosis and resistance to radiation therapy in a variety of cancers, mostly in squamous-cell carcinoma of the head and neck (SCCHN). Blocking of the EGFR signaling results in inhibition of the stimulation of the receptor, cell proliferation, and reduced invasiveness and metastases. The EGFR is, therefore, a prime target for new anticancer therapy in SCCHN.

In certain some embodiments, the present invention relates to the aforementioned method, wherein said SHP2-mediated disorders are cancers selected from, but not limited to: JMML; AML; MDS; B-ALL; neuroblastoma; esophageal; breast cancer; lung cancer; colon cancer; Gastric cancer, Head and Neck cancer.

The compounds of the present invention may also be useful in the treatment of other diseases or conditions related to the aberrant activity of SHP2. Thus, as a further aspect, the invention relates to a method of treatment of a disorder selected from: NS; LS; JMML; AML; MDS; B-ALL; neuroblastoma; esophageal; breast cancer; lung cancer; colon cancer; gastric cancer; head and neck cancer.

### Pharmaceutical Compositions

In general, the compounds of this disclosure will be administered in a therapeutically effective amount by any of the accepted modes of administration for agents that serve similar utilities. Therapeutically effective amounts of compounds this disclosure may range from about 0.01 to about 500 mg per kg patient body weight per day, which can be administered in single or multiple doses. A suitable dosage level may be from about 0.1 to about 250 mg/kg per day; about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount of the compound of this disclosure, i.e., the active ingredient, will depend upon numerous factors such as the severity of the disease to be treated, the age and relative health of the patient, the potency of the compound being utilized, the route and form of administration, and other factors.

In general, compounds of this disclosure will be administered as pharmaceutical compositions by any one of the following routes: oral, systemic (e.g., transdermal, intranasal or by suppository), or parenteral (e.g., intramuscular, intravenous, or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance.

The compositions are comprised of in general, a compound of this disclosure in combination with at least one pharmaceutically acceptable excipient. Acceptable excipients are non-toxic, aid administration, and do not adversely affect the therapeutic benefit of the compound of this disclosure. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol, and various oils, including those of petroleum, animal, vegetable, or synthetic origin, e.g., peanut oil, soybean oil, mineral oil, sesame oil, etc. Preferred liquid carriers, particularly for injectable solutions, include water, saline, aqueous dextrose, and glycols.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

The level of the compound in a formulation can vary within the full range employed by those skilled in the art. Typically, the formulation will contain, on a weight percent (wt. %) basis, from about 0.01-99.99 wt. % of a compound of this disclosure based on the total formulation, with the balance being one or more suitable pharmaceutical excipients.

Pharmaceutically acceptable salts include salts of the active compounds which are prepared with relatively nontoxic acids or bases, depending on the particular substituents found on the compounds described herein. When compounds of the present embodiments contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present embodiments contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19), which is incorporated herein by reference in its entirety for all of its teachings, including without limitation all methods, compounds, compositions, data, and the like, for use with any of the embodiments and disclosure herein. Certain specific compounds of the present embodiments contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present embodiments can exist in unsolvated forms as well as solvated forms, including hydrated forms. In general, the solvated forms are equivalent to unsolvated forms and are encompassed within the scope of the present embodiments. Certain compounds of the present embodiments may exist in multiple crystalline or amorphous forms. In general, all physical forms are equivalent for the uses contemplated by the present embodiments and are intended to be within the scope of the present embodiments.

Certain compounds of the present embodiments possess asymmetric carbon atoms (optical centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present embodiments. The compounds of the present embodiments do not include those which are known in art to be too unstable to synthesize and/or isolate. The present embodiments is meant to include compounds in racemic and optically pure forms. Optically active (R)- and (S)-, or (D)- and (L)-isomers may be prepared using chiral synthons or chiral reagents, or resolved using conventional techniques.

Unless otherwise stated, the compounds of the present embodiments may also contain unnatural proportions of atomic isotopes at one or more of the atoms that constitute such compounds. For example, the compounds of the present embodiments may be labeled with radioactive or stable isotopes, such as for example deuterium (²H), tritium (³H), iodine-125 (¹²⁵I), fluorine-18 (⁸F), nitrogen-15 (¹⁵N), oxygen-17 (¹⁷O), oxygen-18 (¹⁸O), carbon-13 (¹³C), or carbon-14 (¹⁴C). All isotopic variations of the compounds of the present embodiments, whether radioactive or not, are encompassed within the scope of the present embodiments.

In addition to salt forms, the present embodiments provide compounds, which are in a prodrug form. Prodrugs of the compounds described herein are those compounds that readily undergo chemical changes under physiological conditions to provide the compounds of the present embodiments. Additionally, prodrugs can be converted to the compounds of the present embodiments by chemical or biochemical methods in an ex vivo environment. For example, prodrugs can be slowly converted to the compounds of the present embodiments when placed in a transdermal patch reservoir with a suitable enzyme or chemical reagent.

In some embodiments, there are provided pharmaceutical compositions comprising compound I and a pharmaceutically acceptable excipient. In some embodiments, the pharmaceutical compositions are configured as an oral tablet preparation.

The compounds of the present embodiments can be prepared and administered in a wide variety of oral, parenteral, and topical dosage forms. Oral preparations include tablets, pills, powder, dragees, capsules, liquids, lozenges, gels, syrups, slurries, suspensions, etc., suitable for ingestion by the patient. The compounds of the present embodiments can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present embodiments can be administered transdermally. Compound I disclosed herein can also be administered by in intraocular, intravaginal, and intrarectal routes including suppositories, insufflation, powders, and aerosol formulations (for examples of steroid inhalants, see Rohatagi, J Clin. Pharmacol. 35:1187-1193, 1995; Tjwa, Ann. Allergy Asthma Immunol. 75:107-111, 1995), which is incorporated herein by reference in its entirety for all of its teachings, including without limitation all methods, compounds, compositions, data, and the like, for use with any of the embodiments and disclosure herein. Accordingly, the present embodiments also provides pharmaceutical compositions including one or more pharmaceutically acceptable carriers and/or excipients and either compound I, or a pharmaceutically acceptable salt of compound I.

For preparing pharmaceutical compositions from the compounds of the present embodiments, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, surfactants, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”), which is incorporated herein by reference in its entirety for all of its teachings, including without limitation all methods, compounds, compositions, data, and the like, for use with any of the embodiments and disclosure herein.

In powders, the carrier is a finely divided solid, which is in a mixture with the finely divided active component. In tablets, the active component is mixed with the carrier having the necessary binding properties and additional excipients as required in suitable proportions and compacted in the shape and size desired.

The powders, capsules and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers including, but not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations disclosed herein can also be used orally using, for example, push-fit capsules made of gelatin, as well as soft, sealed capsules made of gelatin and a coating such as glycerol or sorbitol. Push-fit capsules can contain compound I mixed with a filler or binders such as lactose or starches, lubricants such as talc or magnesium stearate, and, optionally, stabilizers. In soft capsules, compound I may be dissolved or suspended in suitable liquids, such as fatty oils, liquid paraffin, or liquid polyethylene glycol with or without stabilizers.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame, or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending compound I in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol, or sucrose. These formulations can be preserved by the addition of an antioxidant such as ascorbic acid. As an example of an injectable oil vehicle, see Minto, J. Pharmacol. Exp. Ther. 281:93-102, 1997, which is incorporated herein by reference in its entirety for all of its teachings, including without limitation all methods, compounds, compositions, data, and the like, for use with any of the embodiments and disclosure herein. The pharmaceutical formulations disclosed herein can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The pharmaceutical formulations of compound I disclosed herein can be provided as a salt and can be formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

### Pharmaceutical Dosing

The dosage regimen for the compounds of the present application will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A clinical practitioner can determine and prescribe the effective amount of the drug required to prevent, counter, or arrest the progress of the disease or disorder.

By way of general guidance, the daily oral dosage of each active ingredient, when used for the indicated effects, will range between about 0.001 to about 1000 mg/kg of body weight, preferably between about 0.01 to about 100 mg/kg of body weight per day, and most preferably between about 0.1 to about 20 mg/kg/day. In some embodiments, compound I may be administered at a dose of between about 10 mg/day and about 200 mg/day. In some embodiments, compound I may be administered at a dose of about 10 mg/day, 15 mg/day, 20 mg/day, 25 mg/day, 30 mg/day, 35 mg/day, 40 mg/day, 45 mg/day, 50 mg/day, 55 mg/day, 60 mg/day, 65 mg/day, 70 mg/day, 75 mg/day, 80 mg/day, 85 mg/day, 90 mg/day, 95 mg/day, 100 mg/day, 105 mg/day, 110 mg/day, 115 mg/day, 120 mg/day, 125 mg/day, 130 mg/day, 135 mg/day, 140 mg/day, 145 mg/day, 150 mg/day, 155 mg/day, 160 mg/day, 165 mg/day, 170 mg/day, 175 mg/day, 180 mg/day, 185 mg/day, 190 mg/day, 195 mg/day, or 200 mg/day. The dose may be any value or subrange within the recited ranges.

Depending on the patient's condition and the intended therapeutic effect, the dosing frequency for the therapeutic agent may vary, for example, from once per day to six times per day. That is, the dosing frequency may be QD, i.e., once per day, BID, i.e., twice per day; TID, i.e., three times per day; QID, i.e., four times per day; five times per day, or six times per day. In another embodiment, dosing frequency may be BIW, i.e., twice weekly, TIW, i.e., three times a week, or QIW, i.e. four times a week.

Depending on the patient's condition and the intended therapeutic effect, the treatment cycle may have a period of time where no therapeutic agent is administered. As used herein, “interval administration” refers to administration of the therapeutic agent followed by void days or void weeks. For example, the treatment cycle may be 3 weeks long which includes 2 weeks of dosing of the therapeutic agent(s) followed by 1 week where no therapeutic agent is administered. In some embodiments, the treatment cycle is 4 weeks long which includes 3 weeks of dosing followed by 1 week where no therapeutic agent is administered.

The term “treatment cycle” as used herein, means a pre-determined period of time for administering compound I. In some embodiments, the treatment cycle is a three-week cycle, wherein compound I is administered for the first two weeks of the three-week cycle. In some embodiments, the treatment cycle is a four-week cycle wherein compound I is administered for the first three weeks of the four-week cycle. Typically, the patient is examined at the end of each treatment cycle to evaluate the effect of the therapy. In some embodiments, compound I is administered for multiple cycles (three- or four-week cycles). In some embodiments, compound I is administered for at least one cycle (three- or four-week cycle). In some embodiments, compound I is administered for at least two cycles (three- or four-week cycles). In some embodiments, compound I is administered for at least three cycles (three- or four-week cycles). In some embodiments, compound I is administered for at least four cycles (three- or four-week cycles). In some embodiments, compound I is administered for at least five cycles (three- or four-week cycles). In some embodiments, compound I is administered for at least six cycles (three- or four-week cycles).

In one embodiment, each of the treatment cycle has about 3 or more days. In another embodiment, each of the treatment cycle has from about 3 days to about 60 days. In another embodiment, each of the treatment cycle has from about 5 days to about 50 days. In another embodiment, each of the treatment cycle has from about 7 days to about 28 days. In another embodiment, each of the treatment cycle has 28 days. In one embodiment, the treatment cycle has about 29 days. In another embodiment, the treatment cycle has about 30 days. In another embodiment, the treatment cycle has about 31 days. In another embodiment, the treatment cycle has about a month-long treatment cycle. In another embodiment, the treatment cycle is any length of time from 3 weeks to 8 weeks. In another embodiment, the treatment cycle is any length of time from 3 weeks to 6 weeks. In yet another embodiment, the treatment cycle is 3 weeks. In another embodiment, the treatment cycle is one month. In another embodiment, the treatment cycle is 4 weeks. In another embodiment, the treatment cycle is 5 weeks. In another embodiment, the treatment cycle is 6 weeks. In another embodiment, the treatment cycle is 7 weeks. In another embodiment, the treatment cycle is 8 weeks. The duration of the treatment cycle may include any value or subrange within the recited ranges, including endpoints.

As used herein, the term “co-administration” or “coadministration” refers to administration of (a) an additional therapeutic agent and (b) compound I, or a salt, solvate, ester and/or prodrug thereof, together in a coordinated fashion. For example, the co-administration can be simultaneous administration, sequential administration, overlapping administration, interval administration, continuous administration, or a combination thereof.

In some embodiments, the dosing regimen for compound I is once daily over a continuous 28-day cycle. In some embodiments, the once daily dosing regimen for compound I may be, but is not limited to, 20 mg/day, 25 mg/day, 30 mg/day, 35 mg/day, 40 mg/day, 45 mg/day, 50 mg/day, 55 mg/day, 60 mg/day, or 65 mg/day. Compound I may be administered anywhere from 20 mg to 60 mg once a day. The dose may be any value or subrange within the recited ranges.

In some embodiments, the dosing regimen for compound I is twice daily over a continuous 28-day cycle. In some embodiments, the twice daily dosing regimen for compound I may be, but is not limited to, 10 mg/day, 15 mg/day, 20 mg/day, 25 mg/day, 30 mg/day, 35 mg/day, 40 mg/day, 45 mg/day, 50 mg/day, 55 mg/day, 60 mg/day, 65 mg/day, 70 mg/day, 75 mg/day, 80 mg/day, 85 mg/day, 90 mg/day, 95 mg/day, 100 mg/day, 105 mg/day, 110 mg/day, 115 mg/day, 120 mg/day, 125 mg/day, 130 mg/day, 135 mg/day, 140 mg/day, 145 mg/day, 150 mg/day, 155 mg/day, or 160 mg/day. Compounds of Formula (I) may be administered anywhere from 5 mg to 80 mg twice a day. In some embodiments, compounds of Formula (I) may be administered anywhere from 10 mg/day to 160 mg/day. The dose may be any value or subrange within the recited ranges.

In some embodiments, the dosing regimen for compound I may be once daily, anywhere from 10 mg to 140 mg per day for two weeks, followed by a one week break over a period of 3 weeks. In some embodiments, the dosing regimen for compound I may be once daily, anywhere from 20 mg to 80 mg per day for two weeks, followed by a one week break over a period of 3 weeks.

In some embodiments, the dosing regimen for compound I may be once daily, anywhere from 20 mg to 120 mg per day for two weeks, followed by a one week break in at least one three-week cycle. In some embodiments, the dosing regimen for compound I may be once daily, anywhere from 20 mg to 60 mg per day for two weeks, followed by a one week break in at least one three-week cycle. In some embodiments, the treatment is administered in at least two cycles for a total of at least 6 weeks.

Disclosed herein is a method of treating a disease treatable by inhibition of SHP2 in a patient, comprising administering to the patient a therapeutically effective amount of Compound I:

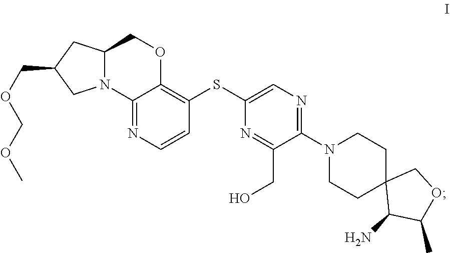 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof, wherein the compound or pharmaceutically acceptable salt thereof, is administered in an amount of 20 mg to 120 mg once a day for two weeks, followed by a one week break in at least one three-week cycle.

Disclosed herein is a method of treating cancer (e.g., colorectal cancer, lung cancer, non-small cell lung cancer, stomach cancer, liver cancer, colon cancer, kidney cancer, breast cancer, head and neck cancer, head and neck squamous cell carcinoma, endometrial carcinoma, pancreatic cancer, pancreatic ductal adenocarcinoma, melanoma, liposarcoma, neuroblastoma, juvenile myelomonocytic leukemia, or acute myeloid leukemia) in a patient, comprising administering to the patient a therapeutically effective amount of Compound I.

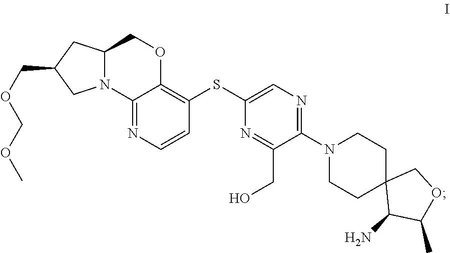 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof, wherein the compound or pharmaceutically acceptable salt thereof, is administered in an amount of 20 mg to 120 mg once a day for two weeks, followed by a one week break in at least one three-week cycle.

In some embodiments, the method further comprises administering cetuximab (Erbitux®, sold by Eli Lilly).

In some embodiments, the dosing regimen for compound I may be 20 mg once daily for two weeks, followed by a one week break in at least one three-week cycle.

In some embodiments, the dosing regimen for compound I may be 25 mg once daily for two weeks, followed by a one week break in at least one three-week cycle.

In some embodiments, the dosing regimen for compound I may be 30 mg once daily for two weeks, followed by a one week break in at least one three-week cycle.

In some embodiments, the dosing regimen for compound I may be 35 mg once daily for two weeks, followed by a one week break in at least one three-week cycle.

In some embodiments, the dosing regimen for compound I may be 40 mg once daily for two weeks, followed by a one week break in at least one three-week cycle.

In some embodiments, the dosing regimen for compound I may be 45 mg once daily for two weeks, followed by a one week break in at least one three-week cycle.

In some embodiments, the dosing regimen for compound I may be 50 mg once daily for two weeks, followed by a one week break in at least one three-week cycle.

In some embodiments, the dosing regimen for compound I may be 55 mg once daily for two weeks, followed by a one week break in at least one three-week cycle.

In some embodiments, the dosing regimen for compound I may be 60 mg once daily for two weeks, followed by a one week break in at least one three-week cycle.

In some embodiments, the dosing regimen for compound I may be 65 mg once daily for two weeks, followed by a one week break in at least one three-week cycle.

In some embodiments, the dosing regimen for compound I may be 70 mg once daily for two weeks, followed by a one week break in at least one three-week cycle.

In some embodiments, the dosing regimen for compound I may be 75 mg once daily for two weeks, followed by a one week break in at least one three-week cycle.

In some embodiments, the dosing regimen for compound I may be 80 mg once daily for two weeks, followed by a one week break in at least one three-week cycle.

In some embodiments, the dosing regimen for compound I may be 85 mg once daily for two weeks, followed by a one week break in at least one three-week cycle.

In some embodiments, the dosing regimen for compound I may be 90 mg once daily for two weeks, followed by a one week break in at least one three-week cycle.

In some embodiments, the dosing regimen for compound I may be 95 mg once daily for two weeks, followed by a one week break in at least one three-week cycle.

In some embodiments, the dosing regimen for compound I may be 100 mg once daily for two weeks, followed by a one week break in at least one three-week cycle.

In some embodiments, the dosing regimen for compound I may be 105 mg once daily for two weeks, followed by a one week break in at least one three-week cycle.

In some embodiments, the dosing regimen for compound I may be 110 mg once daily for two weeks, followed by a one week break in at least one three-week cycle.

In some embodiments, the dosing regimen for compound I may be 115 mg once daily for two weeks, followed by a one week break in at least one three-week cycle.

In some embodiments, the dosing regimen for compound I may be 120 mg once daily for two weeks, followed by a one week break in at least one three-week cycle.

In some embodiments, the dosing regimen for compound I may be twice daily, anywhere from 10 mg to 80 mg twice a day for two weeks, followed by a one week in at least one three-week cycle. In some embodiments, the treatment is administered in at least two cycles for a total of at least six weeks.

In some embodiments, the dosing regimen for compound I may be twice daily, anywhere from 40 mg to 120 mg twice a day for two weeks, followed by a one week break in at least one three-week cycle.

Also disclosed herein is a method of treating a disease treatable by inhibition of SHP2 in a patient, comprising administering to the patient a therapeutically effective amount of Compound I:

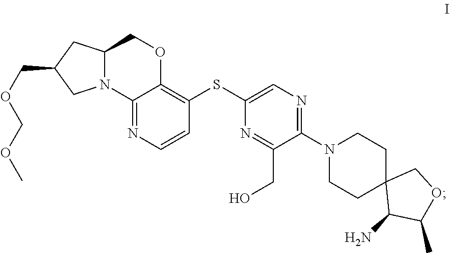 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof, wherein the compound or pharmaceutically acceptable salt thereof, is administered in an amount of 40 mg to 120 mg twice a day for two weeks followed by a one week break in at least one three-week cycle.

Also disclosed herein is a method of treating cancer (e.g., colorectal cancer, lung cancer, non-small cell lung cancer, stomach cancer, liver cancer, colon cancer, kidney cancer, breast cancer, head and neck cancer, head and neck squamous cell carcinoma, endometrial carcinoma, pancreatic cancer, pancreatic ductal adenocarcinoma, melanoma, liposarcoma, neuroblastoma, juvenile myelomonocytic leukemia, or acute myeloid leukemia) in a patient, comprising administering to the patient a therapeutically effective amount of Compound I:

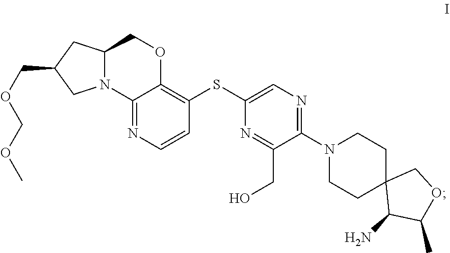 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof, wherein the compound or pharmaceutically acceptable salt thereof, is administered in an amount of 40 mg to 120 mg twice a day for two weeks followed by a one week break in at least one three-week cycle.

In some embodiments, the method further comprises administering cetuximab (Erbitux®, sold by Eli Lilly).

In some embodiments, the dosing regimen for compound I may be twice daily, anywhere from 40 mg to 80 mg twice a day for two weeks, followed by a one week break in at least one three-week cycle.

In some embodiments, the dosing regimen for compound I may be 40 mg twice daily for two weeks, followed by a one week break in at least one three-week cycle.

In some embodiments, the dosing regimen for compound I may be 45 mg twice daily for two weeks, followed by a one week break in at least one three-week cycle.

In some embodiments, the dosing regimen for compound I may be 50 mg twice daily for two weeks, followed by a one week break in at least one three-week cycle.

In some embodiments, the dosing regimen for compound I may be 55 mg twice daily for two weeks, followed by a one week break in at least one three-week cycle.

In some embodiments, the dosing regimen for compound I may be 60 mg twice daily for two weeks, followed by a one week break in at least one three-week cycle.

In some embodiments, the dosing regimen for compound I may be 65 mg twice daily for two weeks, followed by a one week break in at least one three-week cycle.

In some embodiments, the dosing regimen for compound I may be 70 mg twice daily for two weeks, followed by a one week break in at least one three-week cycle.

In some embodiments, the dosing regimen for compound I may be 75 mg twice daily for two weeks, followed by a one week break in at least one three-week cycle.

In some embodiments, the dosing regimen for compound I may be 80 mg twice daily for two weeks, followed by a one week break in at least one three-week cycle.

In some embodiments, the dosing regimen for compound I may be 85 mg twice daily for two weeks, followed by a one week break in at least one three-week cycle.

In some embodiments, the dosing regimen for compound I may be 90 mg twice daily for two weeks, followed by a one week break in at least one three-week cycle.

In some embodiments, the dosing regimen for compound I may be 95 mg twice daily for two weeks, followed by a one week break in at least one three-week cycle.

In some embodiments, the dosing regimen for compound I may be 100 mg twice daily for two weeks, followed by a one week break in at least one three-week cycle.

In some embodiments, the dosing regimen for compound I may be 105 mg twice daily for two weeks, followed by a one week break in at least one three-week cycle.

In some embodiments, the dosing regimen for compound I may be 110 mg twice daily for two weeks, followed by a one week break in at least one three-week cycle.

In some embodiments, the dosing regimen for compound I may be 115 mg twice daily for two weeks, followed by a one week break in at least one three-week cycle.

In some embodiments, the dosing regimen for compound I may be 120 mg twice daily for two weeks, followed by a one week break in at least one three-week cycle.

In some embodiments, the dosing regimen for compound I may be once daily, anywhere from 10 mg to 100 mg per day for three weeks, followed by a one week break in at least one four-week cycle. In some embodiments, the dosing regimen for compound I may be once daily, anywhere from 20 mg to 120 mg per day for three weeks, followed by a one week break in at least one four-week cycle. In some embodiments, the dosing regimen for compound I may be once daily, anywhere from 20 mg to 60 mg per day for three weeks, followed by a one week break in at least one four-week cycle. In some embodiments, the dosing regimen for compound I may be once daily, anywhere from 20 mg to 40 mg per day for three weeks, followed by a one week break in at least one four-week cycle.

Also disclosed herein is a method of treating a disease treatable by inhibition of SHP2 in a patient, comprising administering to the patient a therapeutically effective amount of Compound I:

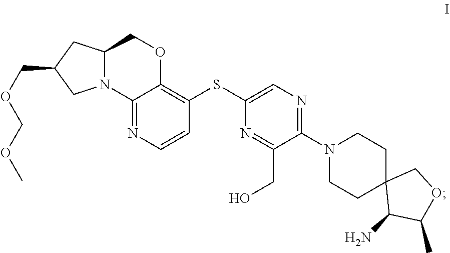 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof, wherein the compound or pharmaceutically acceptable salt thereof, is administered in an amount of 20 mg to 120 mg once a day for three weeks, followed by a one week break in at least one four-week cycle.

Also disclosed herein is a method of treating cancer (e.g., colorectal cancer, lung cancer, non-small cell lung cancer, stomach cancer, liver cancer, colon cancer, kidney cancer, breast cancer, head and neck cancer, head and neck squamous cell carcinoma, endometrial carcinoma, pancreatic cancer, pancreatic ductal adenocarcinoma, melanoma, liposarcoma, neuroblastoma, juvenile myelomonocytic leukemia, or acute myeloid leukemia) in a patient, comprising administering to the patient a therapeutically effective amount of Compound I:

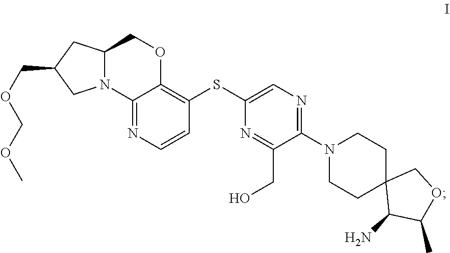 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof, wherein the compound or pharmaceutically acceptable salt thereof, is administered in an amount of 20 mg to 120 mg once a day for three weeks, followed by a one week break in at least one four-week cycle.

In some embodiments, the method further comprises administering cetuximab (Erbitux®, sold by Eli Lilly).

In some embodiments, the dosing regimen for compound I may be 20 mg once daily for three weeks, followed by a one week break in at least one four-week cycle.

In some embodiments, the dosing regimen for compound I may be 25 mg once daily for three weeks, followed by a one week break in at least one four-week cycle.

In some embodiments, the dosing regimen for compound I may be 30 mg once daily for three weeks, followed by a one week break in at least one four-week cycle.

In some embodiments, the dosing regimen for compound I may be 35 mg once daily for three weeks, followed by a one week break in at least one four-week cycle.

In some embodiments, the dosing regimen for compound I may be 40 mg once daily for three weeks, followed by a one week break in at least one four-week cycle.

In some embodiments, the dosing regimen for compound I may be 45 mg once daily for three weeks, followed by a one week break in at least one four-week cycle.

In some embodiments, the dosing regimen for compound I may be 50 mg once daily for three weeks, followed by a one week break in at least one four-week cycle.

In some embodiments, the dosing regimen for compound I may be 55 mg once daily for three weeks, followed by a one week break in at least one four-week cycle.

In some embodiments, the dosing regimen for compound I may be 60 mg once daily for three weeks, followed by a one week break in at least one four-week cycle.

In some embodiments, the dosing regimen for compound I may be 65 mg once daily for three weeks, followed by a one week break in at least one four-week cycle.

In some embodiments, the dosing regimen for compound I may be 70 mg once daily for three weeks, followed by a one week break in at least one four-week cycle.

In some embodiments, the dosing regimen for compound I may be 75 mg once daily for three weeks, followed by a one week break in at least one four-week cycle.

In some embodiments, the dosing regimen for compound I may be 80 mg once daily for three weeks, followed by a one week break in at least one four-week cycle.

In some embodiments, the dosing regimen for compound I may be 85 mg once daily for three weeks, followed by a one week break in at least one four-week cycle.

In some embodiments, the dosing regimen for compound I may be 90 mg once daily for three weeks, followed by a one week break in at least one four-week cycle.

In some embodiments, the dosing regimen for compound I may be 95 mg once daily for three weeks, followed by a one week break in at least one four-week cycle.

In some embodiments, the dosing regimen for compound I may be 100 mg once daily for three weeks, followed by a one week break in at least one four-week cycle.

In some embodiments, the dosing regimen for compound I may be 105 mg once daily for three weeks, followed by a one week break in at least one four-week cycle.

In some embodiments, the dosing regimen for compound I may be 110 mg once daily for three weeks, followed by a one week break in at least one four-week cycle.

In some embodiments, the dosing regimen for compound I may be 115 mg once daily for three weeks, followed by a one week break in at least one four-week cycle.

In some embodiments, the dosing regimen for compound I may be 120 mg once daily for three weeks, followed by a one week break in at least one four-week cycle.

In some embodiments, the dosing regimen for compound I may be twice daily, anywhere from 10 mg to 80 mg twice a day for three weeks, followed by a one week break in at least one four-week cycle. In some embodiments, the treatment is administered in two cycles for a total of eight weeks.

In some embodiments, the dosing regimen for compound I may be twice daily, anywhere from 40 mg to 120 mg twice a day for three weeks, followed by a one week break in at least one four-week cycle.

In some embodiments, the dosing regimen for compound I may be twice daily, anywhere from 40 mg to 80 mg twice a day for three weeks, followed by a one week break in at least one four-week cycle.

Also disclosed herein is a method of treating a disease treatable by inhibition of SHP2 in a patient, comprising administering to the patient a therapeutically effective amount of Compound I:

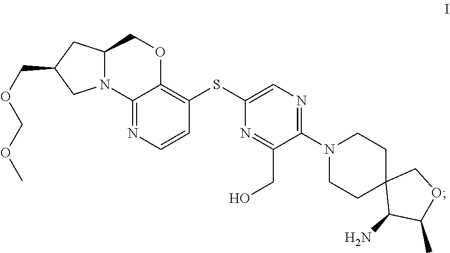 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof, wherein the compound or pharmaceutically acceptable salt thereof, is administered in an amount of 40 mg to 120 mg twice a day for three weeks, followed by a one week break in at least one four-week cycle.

Also disclosed herein is a method of treating cancer (e.g., colorectal cancer, lung cancer, non-small cell lung cancer, stomach cancer, liver cancer, colon cancer, kidney cancer, breast cancer, head and neck cancer, head and neck squamous cell carcinoma, endometrial carcinoma, pancreatic cancer, pancreatic ductal adenocarcinoma, melanoma, liposarcoma, neuroblastoma, juvenile myelomonocytic leukemia, or acute myeloid leukemia) in a patient, comprising administering to the patient a therapeutically effective amount of Compound I.

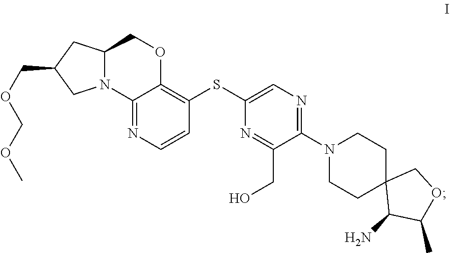 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof, wherein the compound or pharmaceutically acceptable salt thereof, is administered in an amount of 40 mg to 120 mg twice a day for three weeks, followed by a one week break in at least one four-week cycle.

In some embodiments, the method further comprises administering cetuximab (Erbitux®, sold by Eli Lilly).

In some embodiments, the dosing regimen for compound I may be 40 mg twice daily for three weeks, followed by a one week break in at least one four-week cycle.

In some embodiments, the dosing regimen for compound I may be 45 mg twice daily for three weeks, followed by a one week break in at least one four-week cycle.

In some embodiments, the dosing regimen for compound I may be 50 mg twice daily for three weeks, followed by a one week break in at least one four-week cycle.

In some embodiments, the dosing regimen for compound I may be 55 mg twice daily for three weeks, followed by a one week break in at least one four-week cycle.

In some embodiments, the dosing regimen for compound I may be 60 mg twice daily for three weeks, followed by a one week break in at least one four-week cycle.

In some embodiments, the dosing regimen for compound I may be 65 mg twice daily for three weeks, followed by a one week break in at least one four-week cycle.

In some embodiments, the dosing regimen for compound I may be 70 mg twice daily for three weeks, followed by a one week break in at least one four-week cycle.

In some embodiments, the dosing regimen for compound I may be 75 mg twice daily for three weeks, followed by a one week break in at least one four-week cycle.

In some embodiments, the dosing regimen for compound I may be 80 mg twice daily for three weeks, followed by a one week break in at least one four-week cycle.

In some embodiments, the dosing regimen for compound I may be 85 mg twice daily for three weeks, followed by a one week break in at least one four-week cycle.

In some embodiments, the dosing regimen for compound I may be 90 mg twice daily for three weeks, followed by a one week break in at least one four-week cycle.

In some embodiments, the dosing regimen for compound I may be 95 mg twice daily for three weeks, followed by a one week break in at least one four-week cycle.

In some embodiments, the dosing regimen for compound I may be 100 mg twice daily for three weeks, followed by a one week break in at least one four-week cycle.

In some embodiments, the dosing regimen for compound I may be 105 mg twice daily for three weeks, followed by a one week break in at least one four-week cycle.

In some embodiments, the dosing regimen for compound I may be 110 mg twice daily for three weeks, followed by a one week break in at least one four-week cycle.

In some embodiments, the dosing regimen for compound I may be 115 mg twice daily for three weeks, followed by a one week break in at least one four-week cycle.

In some embodiments, the dosing regimen for compound I may be 120 mg twice daily for three weeks, followed by a one week break in at least one four-week cycle.

In some embodiments, the dosing regimen for compound I may be twice daily on days 1 and 2, for four weeks In some embodiments, the dosing amount for compounds of Formula (I) may be, but is not limited to, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, or 160 mg per day. In some embodiments, the dosing amount for compound I may be twice daily, anywhere from 40 mg to 120 mg on days 1 and 2. In some embodiments, the dosing amount for compound I may be twice daily, anywhere from 40 mg to 80 mg on days 1 and 2.

In some embodiments, the dosing regimen for compound I may be once daily on days 1, 2, and 3, for four weeks. In some embodiments, the dosing amount for compounds of Formula (I) may be, but is not limited to, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, or 160 mg per day. In some embodiments, the dosing amount for compound I may be once daily, anywhere from 80 mg to 200 mg on days 1, 2, and 3. In some embodiments, the dosing amount for compound I may be once daily, anywhere from 80 mg to 120 mg on days 1, 2, and 3.

When compound I is administered multiple times a week, the dose may be administered on any day or combination of days within the week. For example, administration three times per week may include administration on days 1, 3, and 5; days 1, 2, and 3; 1, 3, and 5; and so on. Administration two days per week may include administration on days 1 and 2; days 1 and 3; days 1 and 4; days 1 and 5; days 1 and 6; days 1 and 7; and so on.

### Combination

In some embodiments, the methods can include the co-administration of at least one cytotoxic agent. The term “cytotoxic agent” as used herein refers to a substance that inhibits or prevents a cellular function and/or causes cell death or destruction. Cytotoxic agents include, but are not limited to, radioactive isotopes (e.g., At211, I131, I125, Y90, Re186, Re188, Sm153, Bi212, P32, Pb212 and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof.

Examples of cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signaling inhibitors; HDAC inhibitors, proteasome inhibitors; and inhibitors of cancer metabolism.

Chemotherapeutic agents include chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG(geldanamycin), radicicol, lactate dehydrogenase A (LDH-A), fulvestrant (FASLODEX®, AstraZeneca), sunitinib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC 1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5alpha-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γlI and calicheamicin ωlI (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6 azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®); cetuximab (ERBITUX®); panitumumab (VECTIBIX®), rituximab (RITUXAN®), pertuzumab (OMNITARG®, 2C4), trastuzumab (HERCEPTIN®), tositumomab (Bexxar, Corixia), and the antibody drug conjugate, gemtuzumab ozogamicin (MYLOTARG®). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695) which is a recombinant exclusively human-sequence, full-length IgG1 λ antibody genetically modified to recognize interleukin-12 p40 protein.

Chemotherapeutic agent also includes “EGFR inhibitors,” which refers to compounds that bind to or otherwise interact directly with EGFR or its mutant forms and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533) and variants thereof, such as chimerized 225 (C225 or Cetuximab; ERBUTIX®) and reshaped human 225 (H225) (see, WO 96/40210); IMC-11F8, a fully human, EGFR-targeted antibody; antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J Cancer 32A:636-640 (1996)); EMD7200 (matuzumab) a humanized EGFR antibody directed against EGFR that competes with both EGF and TGF-alpha for EGFR binding; human EGFR antibody, HuMax-EGFR; fully human antibodies known as E1.1, E2.4, E2.5, E6.2, E6.4, E2.11, E6. 3 and E7.6. 3 and described in U.S. Pat. No. 6,235,883; MDX-447; and mAb 806 or humanized mAb 806 (Johns et al., J Biol. Chem. 279(29):30375-30384 (2004)). The anti-EGFR antibody may be conjugated with a cytotoxic agent, thus generating an immunoconjugate (see, e.g., EP659,439A2). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA®); PD 183805 (CI 1033, 2-propenamide, N-[4-[(3-chloro-4-fluorophenyl)amino]-7-[3-(4-morpholinyl)propoxy]-6-quinazolinyl]-, dihydrochloride); ZD1839, gefitinib (IRESSA®) 4-(3′-Chloro-4′-fluoroanilino)-7-methoxy-6-(3-morpholinopropoxy)quinazoline); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine). Each of the above-described references is incorporated herein by reference in its entirety for all of its teachings, including without limitation all methods, compounds, compositions, data, and the like, for use with any of the embodiments and disclosure herein.

Chemotherapeutic agents also include “tyrosine kinase inhibitors” including the EGFR-targeted drugs noted in the preceding paragraph; small molecule HER2 tyrosine kinase inhibitor such as TAK165; CP-724,714, an oral selective inhibitor of the ErbB2 receptor tyrosine kinase; dual-HER inhibitors such as EKB-569 which preferentially binds EGFR but inhibits both HER2 and EGFR-overexpressing cells; lapatinib (GSK572016), an oral HER2 and EGFR tyrosine kinase inhibitor; PKI-166; pan-HER inhibitors such as canertinib (CI-1033); Raf-1 inhibitors such as antisense agent ISIS-5132 which inhibit Raf-1 signaling; non-HER targeted TK inhibitors such as imatinib mesylate (GLEEVEC®); multi-targeted tyrosine kinase inhibitors such as sunitinib (SUTENT®); VEGF receptor tyrosine kinase inhibitors such as vatalanib (PTK787/ZK222584); MAPK extracellular regulated kinase I inhibitor CI-1040; quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805; antisense molecules (e.g. those that bind to HER-encoding nucleic acid); quinoxalines (U.S. Pat. No. 5,804,396); tryphostins (U.S. Pat. No. 5,804,396); ZD6474; PTK-787; pan-HER inhibitors such as CI-1033; Affinitac; imatinib mesylate (GLEEVEC®); PKI 166; GW2016; CI-1033; EKB-569; Semaxinib; ZD6474; PTK-787; INC-1C11, rapamycin (sirolimus, RAPAMUNE®); or as described in any of the following patent publications: U.S. Pat. No. 5,804,396; WO 1999/09016; WO 1998/43960; WO 1997/38983; WO 1999/06378; WO 1999/06396; WO 1996/30347; WO 1996/33978; WO 1996/3397; and WO 1996/33980. Each of the above-described references is incorporated herein by reference in its entirety for all of its teachings, including without limitation all methods, compounds, compositions, data, and the like, for use with any of the embodiments and disclosure herein.

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm1⁵³, Bi²¹², P³², Pb²¹² and radioactive isotopes of Lu); miscellaneous investigational agents such as thioplatin, PS-341, phenylbutyrate, ET-18-OCH₃, or famesyl transferase inhibitors (L-739749, L-744832); polyphenols such as quercetin, resveratrol, piceatannol, epigallocatechine gallate, theaflavins, flavanols, procyanidins, betulinic acid and derivatives thereof, autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (RI 1577); orafenib, ABT510; Bcl-2 inhibitor such as oblimersen sodium (GENASENSE®); pixantrone; famesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic, and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

In certain some embodiments, chemotherapeutic agents include, but are not limited to, doxorubicin, dexamethasone, vincristine, cyclophosphamide, fluorouracil, topotecan, interferons, platinum derivatives, taxanes (e.g., paclitaxel, docetaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, trimetrexate, metoprine, cyclosporine, daunorubicin, teniposide, amphotericin, alkylating agents (e.g., chlorambucil), 5-fluorouracil, campthothecin, cisplatin, metronidazole, and imatinib mesylate, among others. In other embodiments, a compound disclosed herein is administered in combination with a biologic agent, such as bevacizumab or panitumumab.

In certain some embodiments, compounds disclosed herein, or a pharmaceutically acceptable composition thereof, are administered in combination with an antiproliferative or chemotherapeutic agent selected from any one or more of abarelix, aldesleukin, alemtuzumab, alitretinoin, allopurinol, altretamine, amifostine, anastrozole, arsenic trioxide, asparaginase, azacitidine, BCG live, bevacuzimab, fluorouracil, bexarotene, bleomycin, bortezomib, busulfan, calusterone, capecitabine, camptothecin, carboplatin, carmustine, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, elotinib, estramustine, etoposide phosphate, etoposide, exemestane, filgrastim, floxuridine, fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

### EXAMPLES

### Biological Assays

### SHP2 Allosteric Inhibition Assay

SHP2 possesses two N-terminal Src homology 2 (SH2) domains, a central protein-tyrosine phosphatase (PTP) domain, and C-terminal tail. At the basal state, SHP2 is auto-inhibited and access of substrates to the catalytic site is blocked by the intermolecular interactions between the SH2 domains and the PTP domain. When bis-tyrosyl-phosphorylated peptides bind to SH2 domain of SHP2, the PTP domain becomes available for substrate recognition and reaction catalysis and SHP2 is allosterically activated. SHP2 catalytic activity can be measured using a fluorogenic artificial substrate DiFMUP.

The phosphatase reactions were carried out at room temperature in 384-well black polystyrene plates (Greiner Bio-One, Cat #784076) using assay buffers containing 60 mM HEPES, pH 7.2, 75 mM NaCl, 75 mM KCl, 1 mM EDTA, 0.05% P-20, and 5 mM DTT.

0.33 nM of SHP2 was co-incubated with of 0.5 μM of bisphos-IRS1 peptide (sequence: H2N-LN(pY)IDLDLV(dPEG8)LST(pY)ASINFQK-amide) and various concentrations of compounds for 30-60 min at room temperature. Then the reaction was initiated by addition of the surrogate substrate DiFMUP (Invitrogen, Cat #D6567, 100 μM final).

The real-time conversion of DiFMUP to DiFMU (6, 8-difluoro-7-hydroxyl-4-methyl-coumarin) was measured every 5 min for 30 min using a microplate reader (CLARIOstar, BMG Labtech) with excitation and emission wavelengths of 340 nm and 450 nm, respectively. Initial reaction rates were determined by linear fitting of the data and the inhibitor dose response curves were analyzed using normalized IC₅₀ regression curve fitting with control-based normalization.

### Pharmaceutical Dosing Study 1

Open-Label Phase 1b/2 Study of Compound of Disclosure in Patients with Advanced or Metastatic Solid Tumors

### Study Design

A compound of the disclosure (e.g., Compound I) in the form of a pharmaceutical composition is administered as a monotherapy to subjects having solid tumors that harbor specific molecular alterations in an open-label, multi-center clinical study. After the screening period, eligible subjects are enrolled and treated with the pharmaceutical composition comprising compound I as monotherapy until disease progression, unacceptable toxicity, or meeting another criterion for stopping treatment.

The study will evaluate the safety and tolerability of escalating doses of the compound of the disclosure when administered as a monotherapy; determine the maximum tolerated dose (MTD) and/or recommended dose (RD) of the compound when administered as a monotherapy; characterize the pharmacokinetic (PK) profile of the compound when administered as a monotherapy; and to evaluate the antitumor activity when administered as a monotherapy.

### Outcome Measures

Primary Outcome Measures to be evaluated: (1) Dose Limiting Toxicities (DLT) (based on toxicities observed) (2) Maximum Tolerated Dose (MTD) (based on toxicities observed) (3) Recommended Dose (RD) (based on toxicities observed) (4) Adverse Events (AE) (incidence and severity of treatment-emergent AEs and serious AEs) (Time frame: assessed up to 24 months from time of first dose) (5) Plasma Concentration (Cmax) (Time Frame: Study Day up to Day 29) (6) Time to Achieve Cmax (Tmax) (Time Frame: Study Day up to Day 29) (7) Area Under the Curve (Area under the plasma concentration-time curve of compound of disclosure) (8) Half-life (Time Frame: Study Day 1 up to Day 29).

Secondary Outcome Measures to be Evaluated: (9) Objective response Rate (ORR) (based on assessment of radiographic imaging per RECIST version 1.1) (time frame: assessed up to 24 months from time of first dose) (10) Duration of Response (DOR) (based on assessment of radiographic imaging per RECIST version 1.1) (11) Time to Response (TTR) (based on assessment of radiographic imaging per RECIST version 1.1) (time frame: assessed up to 24 months from time of first dose).

Other Pre-specified Outcome Measures: (12) Pharmacodynamic Assessment (assessment of phosphorylated ERK (pERK) inhibition in PBMCs or tumor tissue by IHC or immunofluorescence (time frame: assessed up to 24 months from time of first dose.)

### Eligibility

Inclusion Criteria: (1) age≥18 years (2) willing and able to give written informed consent (3) histologically or cytologically confirmed advanced or metastatic solid tumor (4) there is no available standard systemic therapy available for the patient's tumor histology and/or molecular biomarker profile; or standard therapy is intolerable, not effective, or not accessible (5) able to swallow oral medication (6) have Eastern Cooperative Oncology Group Performance Status (ECOG PS) of 0 or 1 (7) adequate cardiovascular, hematological, liver, and renal function and (7) willing to comply with all protocol-required visits, assessments, and procedures.

Exclusion Criteria: (1) previous treatment with a SHP2 inhibitor (2) documented PTPN11 mutations (3) receiving another study therapy or participated in a study of an investigational agent within four weeks of first dose (4) received prior palliative radiation within 7 days of cycle 1, day 1 (5) have primary central nervous system disease or known active CNS metastases and/or carcinomatous meningitis (6) prior surgery or gastrointestinal dysfunction that may affect drug absorption (7) active, clinically signification interstitial lung disease or pneumonitis (8) history of thromboembolic or cerebrovascular events within 12 weeks prior to first dose (9) history or current evidence of retinal vein occlusion (RVO or current risk factors for RVO (10) have any underlying medical condition, psychiatric condition, or social situation that, in the opinion of the Investigator, would comprise study administration as per protocol or compromise the assessment of Aes and (11) pregnant or breastfeeding or expecting to conceive or father children with the projected duration of the trial.

Dose levels for once a day continuous dosing (QD) are 20 mg to 60 mg QD, 40 mg QD, 60 mg QD. Dose levels for twice a day continuous dosing (BID) are 20 mg to 80 mg. Planned dosing schedule for QD or BID is two weeks on/one week off (21 day schedule) and three weeks on/one week off (28 day schedule); three times a week (D1D3D5 TIW) e.g., Day 1, Day 3, and Day 5; twice a day/twice a week e.g., Day 1 and Day 2 (BID-D1D2-BIW).

### Pharmaceutical Dosing Study 2

Open-Label Phase 1b/2 Study of Compound 1 in Combination with Other Anti-Cancer Therapies in Patients with Advanced or Metastatic Solid Tumors Study Design

Compound 1 in the form of a pharmaceutical composition is administered in combination with other cancer therapies (e.g., cetuximab) in subjects having solid tumors that harbor specific molecular alterations in an open-label, multi-center clinical study. After the screening period, eligible subjects are enrolled and treated with the pharmaceutical composition comprising the compound of Formula I and another anti-cancer therapy until disease progression, unacceptable toxicity, or meeting another criterion for stopping treatment.

The study will evaluate the safety and tolerability of escalating doses of Compound 1 when administered in combination with other cancer therapies; determine the maximum tolerated dose (MTD) and/or recommended dose (RD) of Compound 1 when administered in combination with other cancer therapies; characterize the pharmacokinetic (PK) profile of the compound when administered in combination with other cancer therapies; and to evaluate the antitumor activity when administered in combination with other cancer therapies.

### Outcome Measures

Primary Outcome Measures to be evaluated: (1) Dose Limiting Toxicities (DLT) (based on toxicities observed) (2) Maximum Tolerated Dose (MTD) (based on toxicities observed) (3) Recommended Dose (RD) (based on toxicities observed) (4) Adverse Events (AE) (incidence and severity of treatment-emergent AEs and serious AEs) (Time frame: assessed up to 24 months from time of first dose) (5) Plasma Concentration (Cmax) (Time Frame: Study Day up to Day 29) (6) Time to Achieve Cmax (Tmax) (Time Frame: Study Day up to Day 29) (7) Area Under the Curve (Area under the plasma concentration-time curve of compound of disclosure) (8) Half-life (Time Frame: Study Day 1 up to Day 29).

Secondary Outcome Measures to be Evaluated: (9) Objective response Rate (ORR) (based on assessment of radiographic imaging per RECIST version 1.1) (time frame: assessed up to 24 months from time of first dose) (10) Duration of Response (DOR) (based on assessment of radiographic imaging per RECIST version 1.1) (11) Time to Response (TTR) (based on assessment of radiographic imaging per RECIST version 1.1) (time frame: assessed up to 24 months from time of first dose).

Other Pre-specified Outcome Measures: (12) Pharmacodynamic Assessment (assessment of phosphorylated ERK (pERK) inhibition in PBMCs or tumor tissue by IHC or immunofluorescence (time frame: assessed up to 24 months from time of first dose.) Eligibility

Inclusion Criteria: (1) age≥18 years (2) willing and able to give written informed consent (3) histologically or cytologically confirmed advanced or metastatic solid tumor (4) there is no available standard systemic therapy available for the patient's tumor histology and/or molecular biomarker profile; or standard therapy is intolerable, not effective, or not accessible (5) able to swallow oral medication (6) have Eastern Cooperative Oncology Group Performance Status (ECOG PS) of 0 or 1 (7) adequate cardiovascular, hematological, liver, and renal function and (7) willing to comply with all protocol-required visits, assessments, and procedures.

Exclusion Criteria: (1) previous treatment with a SHP2 inhibitor (2) documented PTPN11 mutations (3) receiving another study therapy or participated in a study of an investigational agent within 4 weeks of first dose (4) received prior palliative radiation within 7 days of cycle 1, day 1 (5) have primary central nervous system disease or known active CNS metastases and/or carcinomatous meningitis (6) prior surgery or gastrointestinal dysfunction that may affect drug absorption (7) active, clinically signification interstitial lung disease or pneumonitis (8) history of thromboembolic or cerebrovascular events within 12 weeks prior to first dose (9) history or current evidence of retinal vein occlusion (RVO or current risk factors for RVO (10) have any underlying medical condition, psychiatric condition, or social situation that, in the opinion of the Investigator, would comprise study administration as per protocol or compromise the assessment of Aes and (11) pregnant or breastfeeding or expecting to conceive or father children with the projected duration of the trial.

HPV Negative, Head and Neck Squamous Cell Carcinoma: The preferred dosing schedule is administration of Compound 1 in an amount of 40 mg to 120 mg (40 mg, 60 mg, 80 mg, 100 mg, and 120 mg) twice a day for three weeks, followed by a one week break over a four-week cycle. Additional dosing schedules are as follows: 1) administration of Compound 1 in an amount of 40 mg to 120 mg (40 mg, 60 mg, 80 mg, 100 mg, and 120 mg) twice a day for two weeks, followed by a one week break over a three-week cycle; 2) administration of Compound 1 in an amount of 20 mg to 120 mg (20 mg, 40 mg, 60 mg, 80 mg, 100 mg, and 120 mg) once a day for three weeks, followed by a one week break over a four-week cycle; and 3) administration of Compound 1 in an amount of 20 mg to 120 mg (20 mg, 40 mg, 60 mg, 80 mg, 100 mg, and 120 mg) once a day for two weeks, followed by a one week break over a three-week cycle. Dosing for Cetuximab is dosed weekly or every other week.

wtKRAS wtNRAS/wtBRAF, Colorectal Cancer: The preferred dosing schedule is administration of Compound 1 in an amount of 40 mg to 120 mg (40 mg, 60 mg, 80 mg, 100 mg, and 120 mg) twice a day for three weeks, followed by a one week break over a four-week cycle. Additional dosing schedules are as follows: 1) administration of Compound 1 in an amount of 40 mg to 120 mg (40 mg, 60 mg, 80 mg, 100 mg, and 120 mg) twice a day for two weeks, followed by a one week break over a three-week cycle; 2) administration of Compound 1 in an amount of 20 mg to 120 mg (20 mg, 40 mg, 60 mg, 80 mg, 100 mg, and 120 mg) once a day for three weeks, followed by a one week break over a four-week cycle; and 3) administration of Compound 1 in an amount of 20 mg to 120 mg (20 mg, 40 mg, 60 mg, 80 mg, 100 mg, and 120 mg) once a day for two weeks, followed by a one week break over a three-week cycle. Dosing for Cetuximab is dosed weekly or every other week.

### Formulation Examples

The following are representative pharmaceutical formulations containing a compound of the disclosure.

### Tablet Formulation

The following ingredients are mixed intimately and pressed into single scored tablets.

| | Quantity per |
|---|---|
| Ingredient | tablet (mg) |
| Compound of the disclosure | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| | Quantity per |
|---|---|
| Ingredient | capsule (mg) |
| Compound of the disclosure | 200 |
| lactose spray dried | 148 |
| magnesium stearate | 2 |

### Injectable Formulation

Compound of the disclosure (e.g., compound 1) in 2% HPMC, 1% Tween 80 in DI water, pH 2.2 with MSA, q.s. to at least 20 mg/mL.

What is claimed is:

1. A method of treating a disease treatable by inhibition of SHP2 in a patient, comprising administering to the patient a therapeutically effective amount of Compound I: 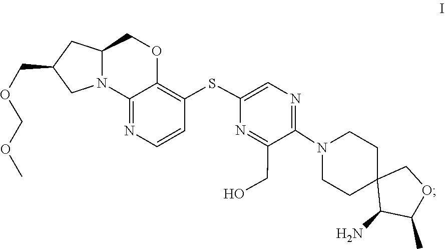 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof.

2. A method of treating a disease treatable by inhibition of SHP2 in a patient, comprising administering to the patient a therapeutically effective amount of Compound I: 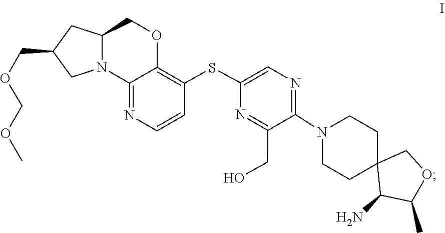 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof; wherein the compound or pharmaceutically acceptable salt thereof, is administered in an amount of 20 mg to 120 mg once a day for two weeks, followed by a one week break in at least one three-week cycle.

3. A method of treating a disease treatable by inhibition of SHP2 in a patient, comprising administering to the patient a therapeutically effective amount of Compound I: 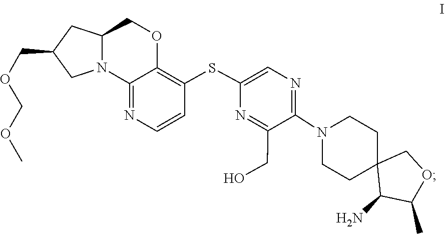 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof; wherein the compound or pharmaceutically acceptable salt thereof, is administered in an amount of 20 mg to 120 mg once a day for three weeks, followed by a one week break in at least one four-week cycle.

4. The method of claim 2 or 3, wherein the compound or pharmaceutically acceptable salt thereof, is administered once a day in an amount of 20 mg to 60 mg.

5. The method of any one of claims 2-4, wherein the compound or pharmaceutically acceptable salt thereof, is administered once a day in an amount of 20 mg to 40 mg.

6. The method of claim 2 or 3, wherein the compound or pharmaceutically acceptable salt thereof, is administered once a day in an amount of 20 mg.

7. The method of claim 2 or 3, wherein the compound or pharmaceutically acceptable salt thereof, is administered once a day in an amount of 25 mg.

8. The method of claim 2 or 3, wherein the compound or pharmaceutically acceptable salt thereof, is administered once a day in an amount of 30 mg.

9. The method of claim 2 or 3, wherein the compound or pharmaceutically acceptable salt thereof, is administered once a day in an amount of 35 mg.

10. The method of claim 2 or 3, wherein the compound or pharmaceutically acceptable salt thereof, is administered once a day in an amount of 40 mg.

11. The method of claim 2 or 3, wherein the compound or pharmaceutically acceptable salt thereof, is administered once a day in an amount of 45 mg.

12. The method of claim 2 or 3, wherein the compound or pharmaceutically acceptable salt thereof, is administered once a day in an amount of 50 mg.

13. The method of claim 2 or 3, wherein the compound or pharmaceutically acceptable salt thereof, is administered once a day in an amount of 55 mg.

14. The method of claim 2 or 3, wherein the compound or pharmaceutically acceptable salt thereof, is administered once a day in an amount of 60 mg.

15. The method of claim 2 or 3, wherein the compound or pharmaceutically acceptable salt thereof, is administered once a day in an amount of 65 mg.

16. The method of claim 2 or 3, wherein the compound or pharmaceutically acceptable salt thereof, is administered once a day in an amount of 70 mg.

17. The method of claim 2 or 3, wherein the compound or pharmaceutically acceptable salt thereof, is administered once a day in an amount of 75 mg.

18. The method of claim 2 or 3, wherein the compound or pharmaceutically acceptable salt thereof, is administered once a day in an amount of 80 mg.

19. The method of claim 2 or 3, wherein the compound or pharmaceutically acceptable salt thereof, is administered once a day in an amount of 85 mg.

20. The method of claim 2 or 3, wherein the compound or pharmaceutically acceptable salt thereof, is administered once a day in an amount of 90 mg.

21. The method of claim 2 or 3, wherein the compound or pharmaceutically acceptable salt thereof, is administered once a day in an amount of 95 mg.

22. The method of claim 2 or 3, wherein the compound or pharmaceutically acceptable salt thereof, is administered once a day in an amount of 100 mg.

23. The method of claim 2 or 3, wherein the compound or pharmaceutically acceptable salt thereof, is administered once a day in an amount of 105 mg.

24. The method of claim 2 or 3, wherein the compound or pharmaceutically acceptable salt thereof, is administered once a day in an amount of 110 mg.

25. The method of claim 2 or 3, wherein the compound or pharmaceutically acceptable salt thereof, is administered once a day in an amount of 115 mg.

26. The method of claim 2 or 3, wherein the compound or pharmaceutically acceptable salt thereof, is administered once a day in an amount of 120 mg.

27. A method of treating a disease treatable by inhibition of SHP2 in a patient, comprising administering to the patient a therapeutically effective amount of Compound I: 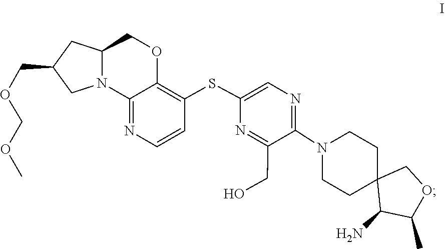 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof; wherein the compound or pharmaceutically acceptable salt thereof, is administered in an amount of 40 mg to 120 mg twice a day for two weeks followed by a one week break in at least one three-week cycle.

28. A method of treating a disease treatable by inhibition of SHP2 in a patient, comprising administering to the patient a therapeutically effective amount of Compound I: 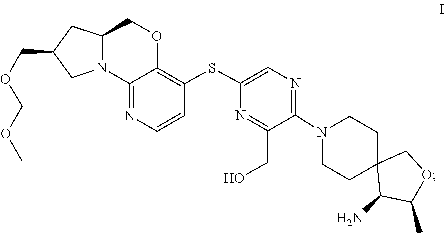 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof; wherein the compound or pharmaceutically acceptable salt thereof, is administered in an amount of 40 mg to 120 mg twice a day for three weeks, followed by a one week break in at least one four-week cycle.

29. The method of claim 27 or 28, wherein the compound or pharmaceutically acceptable salt thereof, is administered twice a day in an amount of 40 mg to 80 mg.

30. The method of claim 27 or 28, wherein the compound or pharmaceutically acceptable salt thereof, is administered twice a day in an amount of 40 mg.

31. The method of claim 27 or 28, wherein the compound or pharmaceutically acceptable salt thereof, is administered twice a day in an amount of 45 mg.

32. The method of claim 27 or 28, wherein the compound or pharmaceutically acceptable salt thereof, is administered twice a day in an amount of 50 mg.

33. The method of claim 27 or 28, wherein the compound or pharmaceutically acceptable salt thereof, is administered twice a day in an amount of 55 mg.

34. The method of claim 27 or 28, wherein the compound or pharmaceutically acceptable salt thereof, is administered twice a day in an amount of 60 mg.

35. The method of claim 27 or 28, wherein the compound or pharmaceutically acceptable salt thereof, is administered twice a day in an amount of 65 mg.

36. The method of claim 27 or 28, wherein the compound or pharmaceutically acceptable salt thereof, is administered twice a day in an amount of 70 mg.

37. The method of claim 27 or 28, wherein the compound or pharmaceutically acceptable salt thereof, is administered twice a day in an amount of 75 mg.

38. The method of claim 27 or 28, wherein the compound or pharmaceutically acceptable salt thereof, is administered twice a day in an amount of 80 mg.

39. The method of claim 27 or 28, wherein the compound or pharmaceutically acceptable salt thereof, is administered twice a day in an amount of 85 mg.

40. The method of claim 27 or 28, wherein the compound or pharmaceutically acceptable salt thereof, is administered twice a day in an amount of 90 mg.

41. The method of claim 27 or 28, wherein the compound or pharmaceutically acceptable salt thereof, is administered twice a day in an amount of 95 mg.

42. The method of claim 27 or 28, wherein the compound or pharmaceutically acceptable salt thereof, is administered twice a day in an amount of 100 mg.

43. The method of claim 27 or 28, wherein the compound or pharmaceutically acceptable salt thereof, is administered twice a day in an amount of 105 mg.

44. The method of claim 27 or 28, wherein the compound or pharmaceutically acceptable salt thereof, is administered twice a day in an amount of 110 mg.

45. The method of claim 27 or 28, wherein the compound or pharmaceutically acceptable salt thereof, is administered twice a day in an amount of 115 mg.

46. The method of claim 27 or 28, wherein the compound or pharmaceutically acceptable salt thereof, is administered twice a day in an amount of 120 mg.

47. The method of any one of claims 1-46, wherein the compound or pharmaceutically acceptable salt thereof is administered for at least two three- or four-week cycles.

48. The method of any one of claims 1-46, wherein the compound or pharmaceutically acceptable salt thereof is administered for at least three three- or four-week cycles.

49. The method of any one of claims 1-46, wherein the compound or pharmaceutically acceptable salt thereof is administered for at least four three- or four-week cycles.

50. The method of any one of claims 1-46, wherein the compound or pharmaceutically acceptable salt thereof is administered for at least five three- or four-week cycles.

51. The method of any one of claims 1-46, wherein the compound or pharmaceutically acceptable salt thereof is administered for at least six three- or four-week cycles.

52. The method of any one of claims 1-51, wherein the compound or pharmaceutically acceptable salt thereof, is formulated as a pharmaceutical composition.

53. The method of any one of claims 1-52, wherein the compound or pharmaceutically acceptable salt thereof, is formulated as an oral composition.

54. The method of any one of claims 1-53, wherein the disease is cancer.

55. The method of claim 54, wherein the cancer is colorectal cancer, lung cancer, non-small cell lung cancer, stomach cancer, liver cancer, colon cancer, kidney cancer, breast cancer, head and neck cancer, head and neck squamous cell carcinoma, endometrial carcinoma, pancreatic cancer, pancreatic ductal adenocarcinoma, melanoma, liposarcoma, neuroblastoma, juvenile myelomonocytic leukemia, or acute myeloid leukemia.

56. The method of any one claims 1-53, wherein the disease is Noonan syndrome or Leopard syndrome.
